(19) United States
(12) Patent Application Publication
(10) Pub. No.: US 2013/0053659 A1
(43) Pub. Date: Feb. 28, 2013

(54) Electric Functional Adhesive Bandage

(71) Applicants: Friedrich Melchert, Gera (DE); Henning Hayn, Stuttgart (DE); Dieter Schwarzmann, Heilbronn (DE)

(72) Inventors: Friedrich Melchert, Gera (DE); Henning Hayn, Stuttgart (DE); Dieter Schwarzmann, Heilbronn (DE)

(73) Assignee: Robert Bosch GmbH, Stuttgart (DE)

(21) Appl. No.: 13/593,585

(22) Filed: Aug. 24, 2012

(30) Foreign Application Priority Data
Aug. 25, 2011 (DE) 10 2011 081 555.4

(51) Int. Cl. A61B 5/145

(52) U.S. Cl. 600/309

(57) ABSTRACT

An electronic functional adhesive bandage includes an adhesive layer and a cover layer, between which an electric energy source, an electric connection element, and an electronic control device are arranged. The electronic functional adhesive bandage also includes a switching element configured to control the power consumption of the electronic control device and to establish an electric connection between the electronic control device, the electric connection element, and the electric energy source.

This application claims priority under 35 U.S.C. §119 to patent application number DE 10 2011 081 555.4 filed on Aug. 25, 2011 in Germany, the disclosure of which is incorporated herein by reference in its entirety.

### BACKGROUND

Adhesive bandages that comprise electronic components, for example in order to monitor human vital parameters, are referred to as electronic functional adhesive bandages. Electronic functional adhesive bandages are currently used for a multiplicity of applications. Known applications include the measurement of EKG or blood oxygen saturation. DE 10 2008 014 652 A1 has also disclosed the use of electronic functional adhesive bandages for detecting sleep apnea.

An electronic functional adhesive bandage comprises an electronic component for signal processing and control, and also, optionally, sensors, actuators and display elements that are integrated in an adhesive bandage. In order to supply the electronic components with electric energy, an independent source of energy is required. An electronic functional adhesive bandage from the prior art is shown in FIG. 1. The electronic components are arranged between an adhesive layer 11, which corresponds to the adhesive layer of a conventional adhesive bandage, and a film as cover layer 12. The adhesive layer 11 consists of a film 111 and a bonding layer 112. Using a wire as connection element 14, a battery as energy source 13 is connected to an electronic control instrument 15 and to a microphone with acoustic horn as sensor 16 for detecting sleep apnea.

Prior to use on the skin of a patient, electronic functional adhesive bandages are activated by the user, i.e. by a patient, a medical practitioner or medical staff, but not by actuating a switch. Due to lack of space and, in particular, from a manufacturing point of view, conventional switches cannot be used in an electronic functional adhesive bandage. This is because to be able to produce electronic adhesive bandages on conventional machines for manufacturing adhesive bandages using roll-to-roll manufacturing, the electronic components must be embedded between a plurality of laminated layers made of plastic or textile materials.

The problem now arises that without a technique for activating the electronic functional adhesive bandage, the functional adhesive bandage is always activated and so energy is used by the electronic components 15, 16 contacting the energy source 13 after production, even without the adhesive bandage being used. The issue with this is that when the functional adhesive bandage is finally used by the user there is no guarantee that there still is sufficient energy available for operating the functional adhesive bandage according to its actual application. Thus, the remaining application duration is reduced the longer the electronic functional adhesive bandage is stored, for example in a pharmacy.

### SUMMARY

The electronic functional adhesive bandage according to the disclosure comprises an adhesive layer and a cover layer, between which an electric energy source, an electric connection element and an electronic control device are arranged. The functional adhesive bandage further comprises a switching element. In one embodiment of the disclosure, this switching element is configured to control the power consumption of the electronic control device. This makes it possible to use an energy-saving state of the control device while the functional adhesive bandage is being stored. When the electronic functional adhesive bandage is used, the switching element is actuated, removing the electronic functional adhesive bandage from the energy-saving state. In a further embodiment of the disclosure, the switching element is configured to establish an electric connection between the electronic control device, the electric connection element and the electric energy source. By separating the energy source from the remaining electronic components until the functional adhesive bandage is finally used, the greatest amount of electric energy is saved and so the functional adhesive bandage can be stored for a much longer time.

When a functional adhesive bandage as per the further embodiment of the disclosure is produced, the energy source is not yet electrically connected to the control device. Then, in order to use the functional adhesive bandage, an electric contact must be established between energy source and control device by mechanical action of the user thereon.

In the first embodiment of the disclosure, in order to save electric energy, provision is made for the control device to be put into a state with low energy uptake, or a stand-by state, using an appropriate switch. As a result of an electric contact being established mechanically, for example by using a pushbutton, the electronics are converted from the stand-by state into the functional state. As a result of using such an energy-saving state, the shelf life of the functional adhesive bandage is increased.

The control device of an electronic functional adhesive bandage is generally a microcontroller with at least one internal oscillator. Thus, according to the disclosure, in the first embodiment it is preferable for the microcontroller to be put into an energy-saving state after the latter was produced and programmed. In this state, the at least one of the internal oscillators is stopped, as a result of which the microcontroller does not continue to operate. Since all that remains switched on is the monitoring of a few external components, e.g. sensors, the power consumption of the electronic functional adhesive bandage drops significantly compared to a conventional functional adhesive bandage. The microcontroller is converted from the energy-saving state into its functional state by actuating a switching element, for example a pushbutton. In the functional state, all internal oscillators are restarted and the microcontroller continues its calculations.

In a further embodiment of the functional adhesive bandage according to the disclosure, the switching element is preferably embodied by the electric energy source moveably mounted in the electronic functional adhesive bandage, wherein the electric energy source is movable between two positions by actuating the switching element. The electric energy source has two poles, which are connected to the control device via two corresponding poles of the electric connection element. In the first position (a), the electric energy source is not electrically connected to the control device with at least one pole. In the second position (b), the electric energy source is electrically connected to the control device with both poles. The use of the energy source as part of the switching element enables a space-saving embodiment of the switching element, which is positioned in the functional adhesive bandage.

According to the disclosure, this switching element is implemented in a number of different ways. In one embodiment, the electronic functional adhesive bandage comprises an attachment element, which fixes one pole of the energy source in position (a) such that it is not connected to the electric connection element and hence not connected to the electronic control device either. In a further embodiment, the electronic functional adhesive bandage has a plurality of fixing elements, which fix both poles of the energy source in position (a) such that they are not connected to the electric connection element and hence not connected to the electronic control device either. In another further embodiment, the energy source is moveably arranged in a guide rail and the electric connection element is arranged at one end of the guide rail. The energy source is displaced in the guide rail between position (a) and position (b). In position (a), the energy source is situated in the guide rail and in position (b) it is pushed out of the guide rail into the electric connection element. The guide rail consists of an electric insulator, with both poles of the electric connection element being situated outside of the guide rail. In a further embodiment, the energy source is moveably arranged in an electrically conductive guide rail, with the guide rail forming one pole of the electric connection element and the other pole being situated outside of the guide rail. The energy source is displaceable in the guide rail between position (a) and position (b). In position (a), the energy source is completely within the guide rail and in position (b) the energy source is partly pushed out of the guide rail such that it contacts both the guide rail and the pole of the electric connection element arranged outside of the guide rail. In another, different embodiment, the electronic functional adhesive bandage comprises a clamping device, which has two clamping elements. In position (a), the electric energy source is arranged in the first clamping element and, in position (b), the electric energy source is arranged in the second clamping element.

In all of these embodiments, it is possible to establish an electric contact at both poles of the energy source by exerting mechanical pressure on one side of the energy source. In at least one embodiment, the mechanical pressure is exerted by a user pushing a finger on a marked point on the electronic functional adhesive bandage. In these embodiments, the switching element is preferably configured such that it is no longer possible to remove the energy source from position (b).

This prevents the power supply of the control device from being inadvertently interrupted when the electronic functional adhesive bandage is being used.

The electric energy source is preferably a battery, more particularly a button cell.

In a further embodiment, the energy source is fixedly incorporated in the electronic functional adhesive bandage. The switching element is embodied as a pushbutton with a feeler, which is arranged in the electric connection element between the control device and the electric energy source. In this embodiment, the electric connection element is interrupted between the control device and the electric energy source. The pushbutton is configured to irreversibly eliminate this interruption by being actuated. The pushbutton is preferably embodied as a single-gang switch in order to prevent the energy supply of the control device from being inadvertently interrupted when the electronic functional adhesive bandage is being used. However, according to the disclosure, it is also possible to use a multiple-gang switch as switching element. A pushbutton constitutes a space-saving embodiment of the switching element according to the disclosure.

According to the disclosure, it is preferable for the point on which the user pushes to activate the functional adhesive bandage to be marked on the outer side of the adhesive bandage, for example by color or by a label or an indentation/bulge. This marking is applied to the cover layer.

### BRIEF DESCRIPTION OF THE DRAWINGS

Exemplary embodiments of the disclosure are illustrated in the drawings and explained in more detail in the following description.

In the drawings:

FIG. 1 shows an electronic functional adhesive bandage according to the prior art;

FIG. 2 shows a program flowchart for an electronic functional adhesive bandage as per a first embodiment of the present disclosure;

FIG. 3a shows the switching element of a second embodiment of the present disclosure, in which the electric connection between energy source and control device is interrupted;

FIG. 3b shows the switching element of the second embodiment of the present disclosure, in which there is an electric connection between energy source and control device;

FIG. 4a shows the switching element of a third embodiment of the present disclosure, in which the electric connection between energy source and control device is interrupted;

FIG. 4b shows the switching element of the third embodiment of the present disclosure, in which there is an electric connection between energy source and control device;

FIG. 4c shows a plan view of the switching element of the third embodiment of the present disclosure as per FIG. 4b;

FIG. 4d shows an electronic functional adhesive bandage with a switching element as per FIG. 4a;

FIG. 5a shows the switching element of a fourth embodiment of the present disclosure, in which there is no electric connection between energy source and control device;

FIG. 5b shows the switching element of the fourth embodiment of the present disclosure, in which there is an electric connection between energy source and control device;

FIG. 6a shows the switching element of a fifth embodiment of the present disclosure, in which the electric connection between energy source and control device is interrupted;

FIG. 6b shows a plan view of the switching element of the fifth embodiment of the present disclosure as per FIG. 6a;

FIG. 6c shows the switching element of the fifth embodiment of the present disclosure, in which there is an electric connection between energy source and control device;

FIG. 6d shows a plan view of the switching element of the fifth embodiment of the present disclosure as per FIG. 6c;

FIG. 7a shows the switching element of a sixth embodiment of the present disclosure, in which the electric connection between energy source and control device is interrupted;

FIG. 7b shows the switching element of the sixth embodiment of the present disclosure, in which there is an electric connection between energy source and control device;

FIG. 7c shows an electronic functional adhesive bandage with a switching element as per FIG. 7a;

FIG. 8a shows the switching element of a seventh embodiment of the present disclosure, in which the electric connection between energy source and control device is interrupted;

FIG. 8b shows the switching element of the seventh embodiment of the present disclosure, in which there is an electric connection between energy source and control device;

FIG. 9a shows the switching element of an eighth embodiment of the invention, in which the electric connection between energy source and control device is interrupted; and

FIG. 9b shows the switching element of the eighth embodiment of the invention, in which there is an electric connection between energy source and control device.

### DETAILED DESCRIPTION

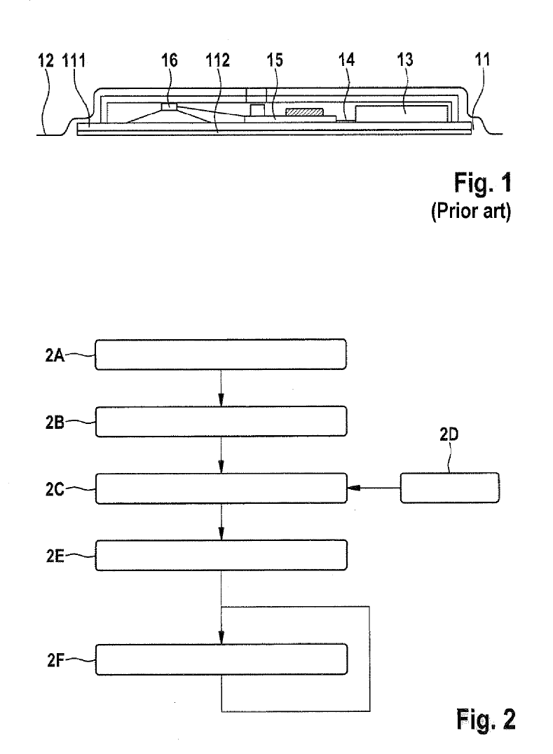

In a first embodiment of the disclosure, the configuration of the electronic functional adhesive bandage corresponds to that of a conventional electronic functional adhesive bandage as illustrated in FIG. 1. A microcontroller 15, which comprises a pushbutton configured to terminate an energy-saving state of the microcontroller, is used as an electronic control device 15. FIG. 2 illustrates how such an electronic functional adhesive bandage is used to save energy during storage. The microcontroller is initially produced and programmed (2A). Then it is put into an energy-saving state (2B). In this state, all internal oscillators of the microcontroller are stopped, as a result of which the microcontroller does not continue to operate. As a result, the energy consumption of the microcontroller is significantly reduced because all that is switched on is the monitoring of a few external components. The microcontroller awaits activation by a pushbutton being pushed (2C). As soon as this pushbutton is pushed (2D), there is an external interrupt. As a result, all internal oscillators are restarted (2E). The microcontroller continues its calculations (2F). The point the user pushes to activate the microcontroller is labeled, e.g. by color, on the cover film 12 of the electronic functional adhesive bandage.

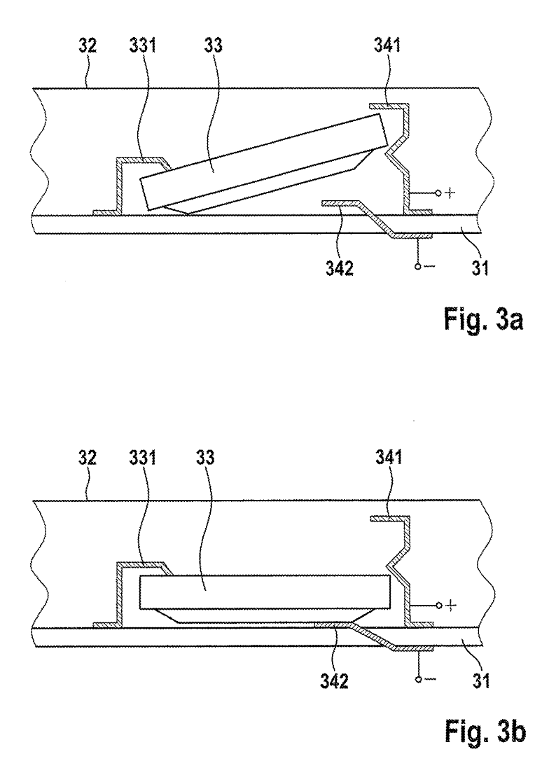

In a second embodiment of the present disclosure, the energy source is fixed on one side in position (a) such that no electric contact to the control device is established on one pole of the energy source. This is illustrated in FIG. 3a. A button cell 33 is arranged between an adhesive layer 31 and a cover layer 32. On one side, said button cell is connected to the adhesive layer 31 by a connection element 331. On the other side, it rests on a pole 341 of the electric connection element. This pole 341 is embodied as bent metal leaf, which fixes the battery in a position in which it is at an angle with respect to the adhesive layer 31. To this end, part of the pole 341 is embodied as attachment element. Parallel to the adhesive layer 31, the second pole 342 of the electric connection element is arranged such that it has no contact with the battery 33. As a result of mechanical pressure on the upper side of the battery 33, the battery 33 is pushed downward and brought into a position (b), in which it is aligned parallel to the adhesive layer 31 and rests thereon. As a result, an electric contact is established with both poles 341, 342 of the electric connection element. This is illustrated in FIG. 3b. The metal leaf of the pole 341 is shaped such that the battery 33 is respectively fixed in the position as per FIG. 3a and in the position as per FIG. 3b. The metal leaf of the pole 341 acts as a leaf spring with an element configured to latch the battery 33.

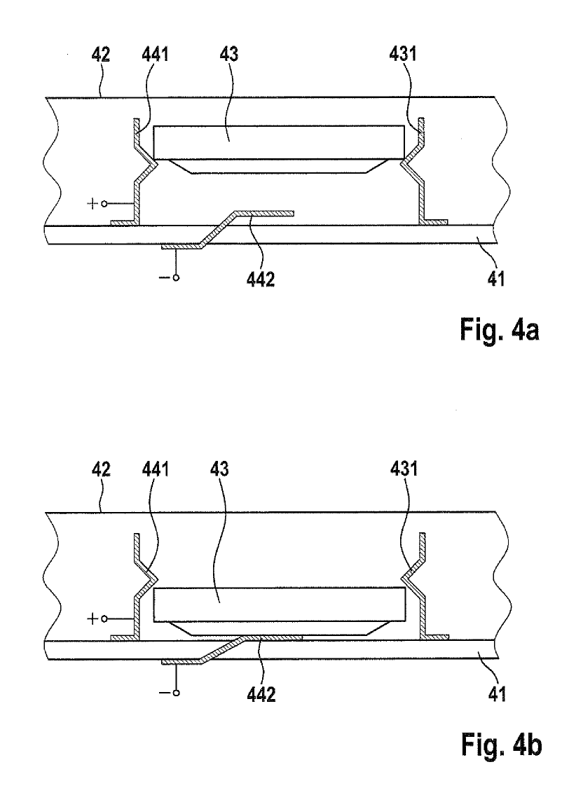

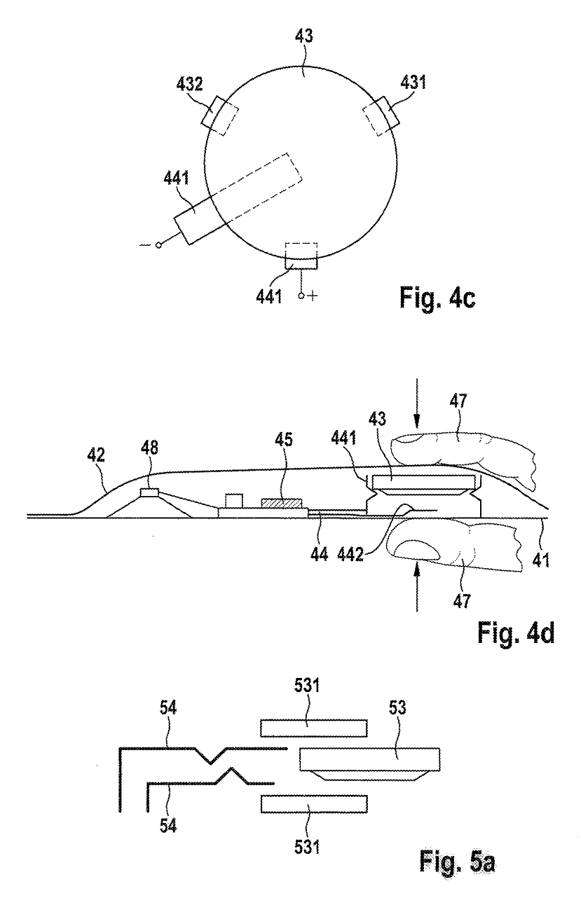

A third embodiment of the present disclosure is illustrated in FIGS. 4a to 4d. A button cell as energy source 43 is arranged between the adhesive layer 41 and the cover layer 42 of the functional adhesive bandage. FIG. 4a shows how said button cell is fixed by three bent metal leaves 431, 432 (shown in FIG. 4c), 441 in a position (a), in which it is arranged parallel and at a distance to the adhesive layer 41. One of these metal leaves is embodied as a pole 441 of the electric connection element. Two further metal leaves 431, 432 (shown in FIG. 4c) serve as fixing elements, which are unable to establish electric contact to the control device 45 of the electronic functional adhesive bandage. The second pole 442 of the electric connection element is arranged on the surface of the adhesive layer 41. As a result of mechanical pressure on the upper side of the battery 43, the battery 43 is pushed downward such that it rests on the adhesive layer 41. In doing so, the contact to the first pole 441 of the electric connection element is maintained and, in addition, contact is established with the second pole 442 of the electric connection element. This is illustrated in FIG. 4b. The battery 43 is fixed in this position (b) by the bent metal leaves 431, 432 (shown in FIG. 4c), 441. FIG. 4d illustrates how a switching element, which includes the battery 43 and the fixing elements 431, 432 (shown in FIG. 4c) and the electric poles 441, 442, is embedded into an electronic functional adhesive bandage. The electronic functional adhesive bandage comprises an adhesive layer 41, a cover layer 42, a control device 48 and a microphone with an acoustic horn as sensor 46 for detecting sleep apnea. A user actuates the switching element with his fingers 47.

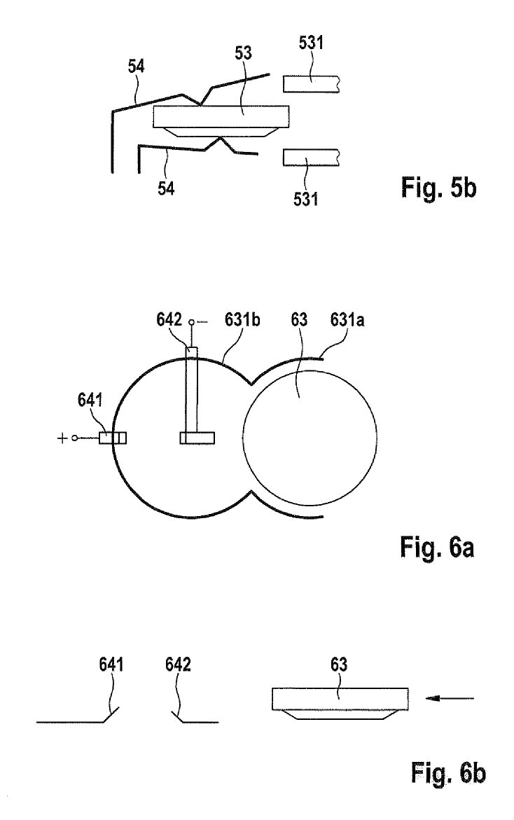

A fourth embodiment of the present disclosure is illustrated in FIGS. 5a and 5b. A button cell as energy source 53 is arranged in a non-conductive guide rail 531. In a first position (a), the battery 53 is at a distance from the electric connection element 54, which is arranged at the end of the guide rail 531. This is illustrated in FIG. 5a. As a result of mechanical pressure from the side, the battery 53 is displaced along the guide rail 531. The battery 53 is pushable out of the guide rail 531 into a position (b). FIG. 5b shows how contact with the battery 53 is established by two metal leaves of the electric connection element 54 in this position (b). As a result of the shape of the metal leaves of the electric connection element, the battery 53 is fixed in position (b).

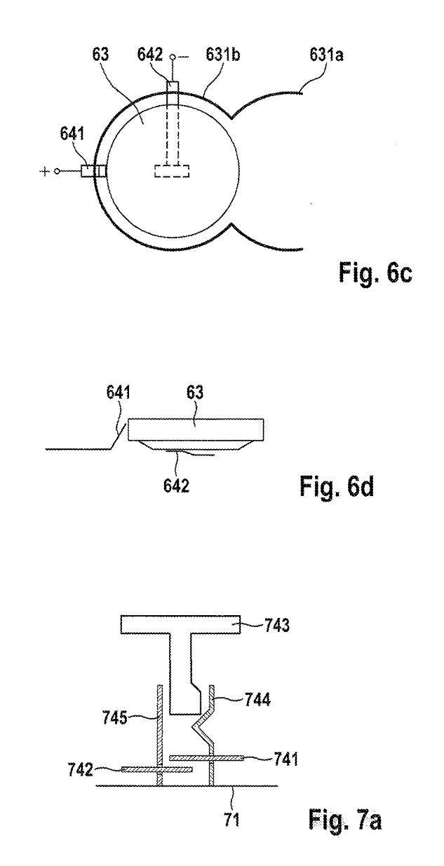

FIGS. 6a to 6d show a fifth embodiment of the present disclosure. A button cell as energy source 63 is arranged in a clamping device. The clamping device comprises two clamping elements 631a, 631b. FIGS. 6a and 6b illustrate how the battery 63 in a position (a) is arranged in the first clamping element 631a. The battery 63 has no contact with the electric connection element with the poles 641, 642. As a result of lateral pressure on the battery 63, the battery 63 is moved into the second clamping element 631b of the clamping device. In this position (b), which is shown in FIGS. 6c and 6d, the battery 63 contacts the two poles 641, 642 of the electric connection element. In the lateral views 6b and 6d, the clamping element is not illustrated in each case in order to clarify the interaction between the battery 63 and the poles 641, 642.

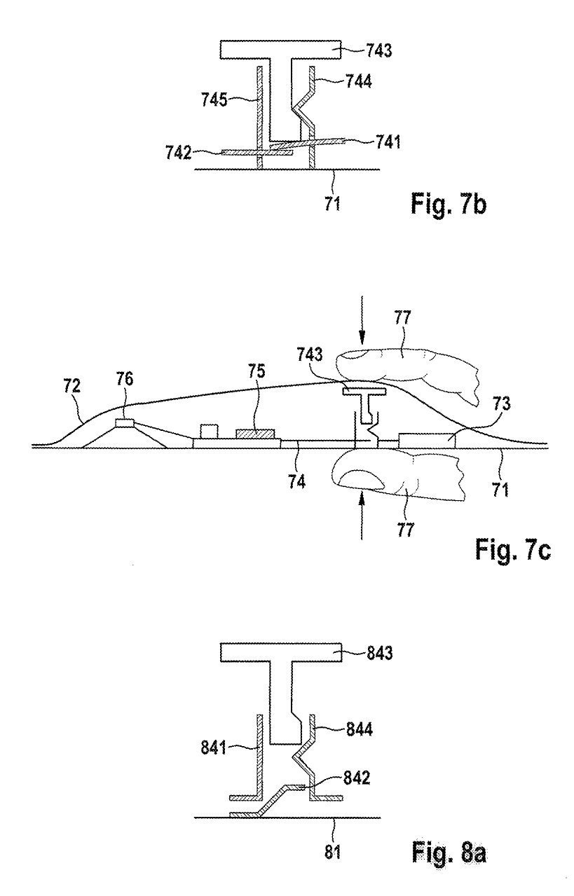

In a sixth embodiment of the present disclosure, the energy source is fixedly incorporated into the electronic functional adhesive bandage, as known from conventional electronic functional adhesive bandages. FIG. 7a shows that the electric connection element 74 (shown in FIG. 7c) is interrupted such that there is no connection between the energy source and the electronic control device. For a switching element, a pushbutton is provided which is configured to interconnect two poles 741, 742 of the electric connection element. Here, a bent metal leaf 744 and a further metal leaf 745 fix the feeler 743 of the pushbutton in a position in which it is situated above the two poles 741, 742. The two poles 741, 742 are embodied as metal leaves, which are arranged parallel to one another and to the adhesive layer 71 of the electronic functional adhesive bandage. As a result of mechanical pressure onto the switching element from above, the feeler 743 is pushed downward into position (b) and the feeler 743 pushes the two metal leaves 741, 742 against one another such that electric contact is established therebetween. This is illustrated in FIG. 7b. At the end facing the adhesive layer 71, the pushbutton 743 has a thickening. As a result, the feeler 743 is fixed in position (b) by the bent metal leaf 744. FIG. 7c shows an electronic functional adhesive bandage which contains such a pushbutton as switching element. Arranged between the adhesive layer 71 and a cover layer 72 there are an energy source 73, an electronic control element 75 and a microphone with acoustic horn as a sensor 76. The connection between the energy source 73 and the electronic control device 75 is established by the connection element 74 when the pushbutton 743 is actuated by the fingers 77 of the user.

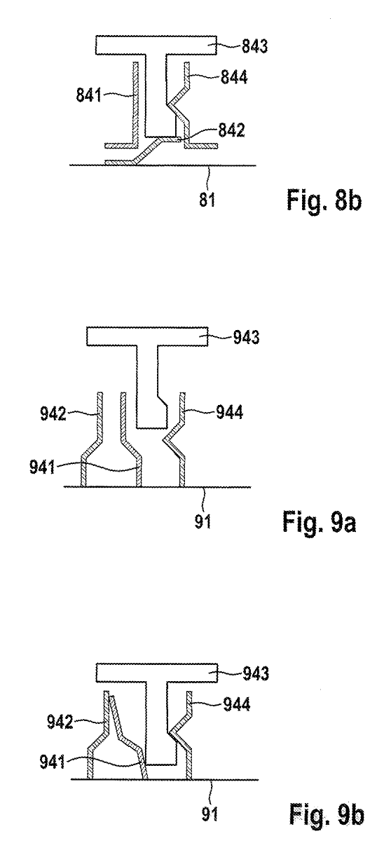

A seventh embodiment of the present disclosure is illustrated in FIGS. 8a and 8b. In contrast to the sixth embodiment, one of the two metal leaves which hold the feeler 843 is connected to the electric connection element. It forms the first pole 841 of the electric connection element. The second pole 842 of the electric connection element is, like in the sixth embodiment, embodied as metal leaf which is arranged parallel to the adhesive layer 81 of the electronic functional adhesive bandage. A bent metal leaf 844 fixes the feeler 843 in position (a), in which it is situated above the pole 842. The feeler 843 consists of an electrically conductive material. As a result of mechanical pressure on the switching element from above, the feeler 843 is pushed downward and the feeler 843 touches the pole 842 while, at the same time, remaining in contact with the first pole 841 such that electric contact is established between the poles 841, 842. This is illustrated in FIG. 8b. Like in the sixth embodiment, the pushbutton 843 has a thickening at the end facing the adhesive layer 81 and is fixed in position (b) by the bent metal leaf 844.

FIGS. 9a and 9b show an eighth embodiment of the present disclosure. FIG. 9a shows that the electric connection element is interrupted, and so there is no connection between the energy source and the electronic control device. For a switching element, a pushbutton is provided which is configured to interconnect two poles 941, 942 of the electric connection element. Both poles 941, 942 are embodied as metal leaves. The pole 941 is in contact with the feeler 943 of the pushbutton. Here, the feeler 943 is fixed in a position (a) by the pole 941 and a bent metal leaf 944. As a result of mechanical pressure on the switching element from above, the feeler 943 is pushed downward into position (b). In the process, the feeler 943 deforms the pole 941 such that it is brought into electric contact with the pole 942. This is illustrated in FIG. 9b. Like in the sixth and seventh embodiments, the pushbutton 943 has a thickening at the end facing the adhesive layer 91 and is fixed in position (b) by the bent metal leaf 944.

In the electronic functional adhesive bandage according to the present disclosure as per the second to eighth embodiments, the voltage supply of the electronic components is only put into operation when the adhesive bandage is in fact put to use. This ensures that there is always a sufficient amount of energy for operating the functional adhesive bandage.

What is claimed is:

1. An electronic functional adhesive bandage, comprising:
an adhesive layer;
a cover layer;
an electric energy source arranged between the adhesive layer and the cover layer;
an electric connection element arranged between the adhesive layer and the cover layer;
an electronic control device arranged between the adhesive layer and the cover layer; and
a switching element configured to control power consumption of the electronic control device and configured to establish an electric connection between the electronic control device, the electric connection element, and the electric energy source.

2. The electronic functional adhesive bandage of claim 1, wherein:
the electric energy source includes a first pole and a second pole and is moveably mounted in the electronic functional adhesive bandage; and
the switching element is configured to move the electric energy source between a first position, in which at least one of the first pole and the second pole is not electrically connected to the electronic control device, and a second position, in which both the first pole and the second pole are electrically connected to the electronic control device.

3. The electronic functional adhesive bandage of claim 2, further comprising:
an attachment element configured to fix the electric energy source in the first position such that at least one of the first pole and the second pole is not connected to the electric connection element.

4. The electronic functional adhesive bandage of claim 2, further comprising:
a plurality of fixing elements configured to fix the electric energy source in the first position such that neither of the first pole and the second pole is connected to the electric connection element.

5. The electronic functional adhesive bandage of claim 1, further comprising:
a guide rail, wherein:
the electric energy source is moveably arranged in the guide rail; and
the electric connection element is arranged at an end of the guide rail.

6. The electronic functional adhesive bandage of claim 2, further comprising:
a clamping device having a first clamping element and a second clamping element, wherein:
the electric energy source is in the first position when arranged in the first clamping element; and
the electric energy source is in the second position when arranged in the second clamping element.

7. The electronic functional adhesive bandage of claim 1, wherein:
the switching element is a pushbutton having a feeler, the feeler arranged in the electric connection element between the electronic control device and the electric energy source.

8. The electronic functional adhesive bandage of claim 1, wherein:
the electric connection element includes an interruption between the electronic control device and the electric energy source; and
the switching element is configured to irreversibly eliminate the interruption.

9. The electronic functional adhesive bandage of claim 1, further comprising:
at least one oscillator provided in the electronic control device, the at least one oscillator configured to be started by actuation of the switching element.

10. The electronic functional adhesive bandage of claim 1, wherein:
a position of the switching element is marked on an outer side of the cover layer.
